(19) United States
(12) Patent Application Publication
(10) Pub. No.: US 2023/0101619 A1
(43) Pub. Date: Mar. 30, 2023

(54) ELECTRICAL BICYCLE ("E-BIKE") DETECTOR FOR ENERGY EXPENDITURE ESTIMATION

(71) Applicant: Apple Inc., Cupertino, CA (US)

(72) Inventors: Robin Guers, San Jose, CA (US); Tue Doan, San Jose, CA (US); Rodney Jeung, San Francisco, CA (US); Ziyi Zhou, Santa Clara, CA (US); Daniel J. Perry, San Jose, CA (US)

(21) Appl. No.: 17/478,847

(22) Filed: Sep. 17, 2021

(51) Int. Cl. A63B 24/00; A61B 5/22; A61B 5/024; A61B 5/11; A61B 5/00; G06N 7/00

(52) U.S. Cl. CPC A63B 24/0062; A61B 5/222; A61B 5/681; A61B 5/1112; A61B 5/1118; A61B 5/02438; A61B 5/4884; G06N 7/005; A63B 2024/0065

(57) ABSTRACT

Embodiments are disclosed for an electrical bicycle detector for energy expenditure estimation. In an embodiment, a method comprises: determining heart rate energy expenditure of a user wearing or holding the device; determining work rate energy expenditure of a user wearing or holding the device; determining a probability that the user is riding an electrical bike based on the heart rate energy expenditure and the work rate energy expenditure; determining whether or not the probability meets a condition corresponding to a threshold probability; and in accordance with the probability meeting the condition corresponding to the threshold probability: adjusting the work rate energy expenditure; and generating fitness data based at least on the adjusted work rate energy expenditure.

### TECHNICAL FIELD

This disclosure relates generally to fitness monitoring using wearable devices.

### BACKGROUND

Modern wearable devices (e.g., smart watches, fitness bands) are often used by individuals during fitness activities to determine their energy expenditure during the fitness activity. Some wearable devices include inertial sensors (e.g., accelerometers, angular rate sensors) that are used to estimate a work rate (WR) based metabolic equivalent of task (MET) for the user wearing the device. Some wearable devices also include a heart rate (HR) sensor that provides HR data that can be used with user estimated VO₂ MAX (maximal oxygen consumption) and other data (e.g., users weight, age) to estimate the user’s HR based MET. The WR MET and HR MET are combined in some suitable manner (e.g., averaged) to determine the energy expenditure of the user.

An electrical bicycle (“e-bike”) is a bicycle with an integrated electric motor used to assist the rider’s pedal-power. Because of this propulsion assistance, the wearable device overestimates WR METs, resulting in an inaccurate calculation of the user’s energy expenditure.

### SUMMARY

Embodiments are disclosed for an e-bike detector for energy expenditure estimation.

In an embodiment, a method comprises: determining a heart rate energy expenditure of a user wearing or holding the device; determining work rate energy expenditure of a user wearing or holding the device; determining a probability that the user is riding an electrical bike based on the heart rate energy expenditure and the work rate energy expenditure; determining whether or not the probability meets a condition corresponding to a threshold probability; and in accordance with the probability meeting the condition corresponding to the threshold probability: adjusting the work rate energy expenditure; and generating fitness data based at least on the adjusted work rate energy expenditure.

Other embodiments can include an apparatus, computing device and non-transitory, computer-readable storage medium.

Particular embodiments disclose herein provide one or more of the following advantages. The energy expenditure of a user is more accurately calculated by detecting whether or not the user is riding an e-bike. When an e-bike is detected by an e-bike detection model, a WR MET output by a WR energy expenditure model is compensated (e.g., scaled down by a weighting factor).

The details of one or more implementations of the subject matter are set forth in the accompanying drawings and the description below. Other features, aspects and advantages of the subject matter will become apparent from the description, the drawings and the claims.

### BRIEF DESCRIPTION OF THE DRAWINGS

FIG. 1 illustrates a typical e-bike, according to an embodiment.

FIGS. 2A and 2B show e-bike models for flat and incline surfaces, respectively, according to an embodiment.

FIGS. 3A-3C illustrate sensor the overestimation of WRMETs when the user is riding an e-bike, according to an embodiment.

FIG. 4 is a block diagram of a system for determining e-bike energy expenditure, according to an embodiment.

FIG. 5 is a flow diagram of a process of e-bike energy expenditure, according to an embodiment.

FIG. 6 is example wearable device architecture for implementing the features and processes described in reference to FIGS. 1-5.

### DETAILED DESCRIPTION

### Overview

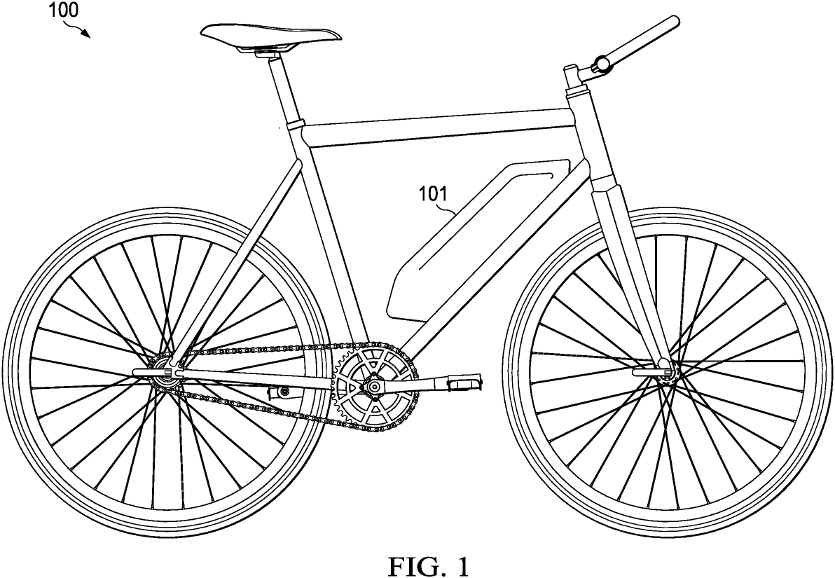

FIG. 1 illustrates a typical e-bike 100, according to an embodiment. E-bike 100 differs from a conventional road bike in that it includes an integrated electric motor 101 that assists the user’s pedal-power. Because e-bike 100 requires less effort from the user it is ideal for users who want to get back into cycling and to cover more distance with less pedal-power.

In an embodiment, the device is a smartwatch or fitness band worn on the wrist of the user or other body part (e.g., waist, chest, leg).

In an embodiment, determining a probability that the user is riding an electrical bicycle based on the heart rate energy expenditure and the work rate energy expenditure includes evaluating a probability function that includes a ratio of work rate energy expenditure and heart rate energy expenditure.

In an embodiment, the probability function is also a function of incline data indicating that the user is riding a bike or electrical bike on an incline.

In an embodiment, the probability function is given by:

$$ln \frac{p}{1 - p} = {β}_{0} + {β}_{1} {x}_{1} + {β}_{2} {x}_{2} + {β}_{3} {x}_{1} {x}_{2} ,$$

where p is the probability of being an electrical bicycle, x₁ is the ratio between work rate energy expenditure and heart rate energy expenditure and x₂ is a boolean value of “1” for being an incline or “0” for being flat.

In an embodiment, determining whether or not the probability meets a condition corresponding to a threshold probability, further comprises: computing the probability over one or more sliding windows of work rate energy expenditure and heart rate energy expenditure values; and determining a percentage of measurement epochs in the one or more windows having a probability that meets the condition corresponding to the threshold probability.

In an embodiment, the method further comprises: determining that an electrical bicycle is detected based on two or more sliding windows having a percentage of measurement epochs having a probability that meets the condition corresponding to the threshold probability

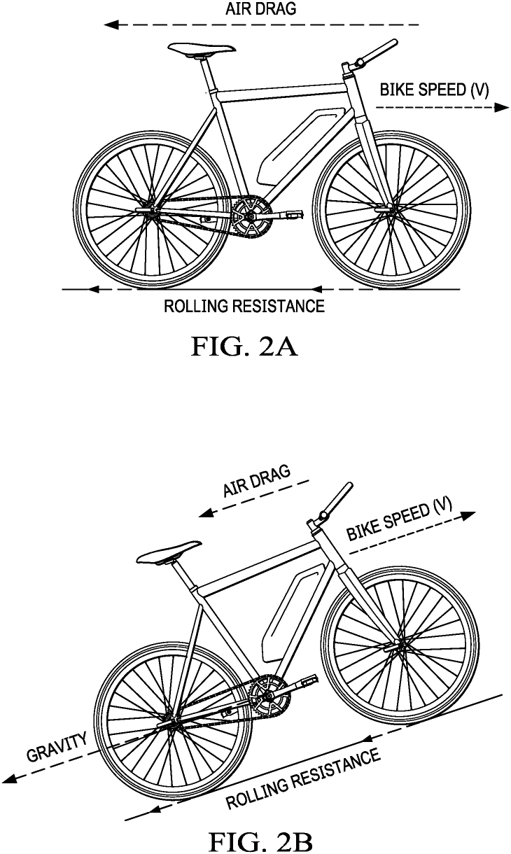

FIGS. 2A and 2B show e-bike models for flat and incline surfaces, respectively, according to an embodiment. When e-bike 100 is traveling on a flat surface it is subjected to wind resistance (air drag) and rolling resistance (friction), which can slow the speed (V) of e-bike 100, as shown in FIG. 2A. When e-bike 100 is traveling on an inclined surface e-bike 100 is subjected to wind resistance, rolling resistance (friction) and gravity, which can slow the speed (V) of e-bike 100, as shown in FIG. 2B.

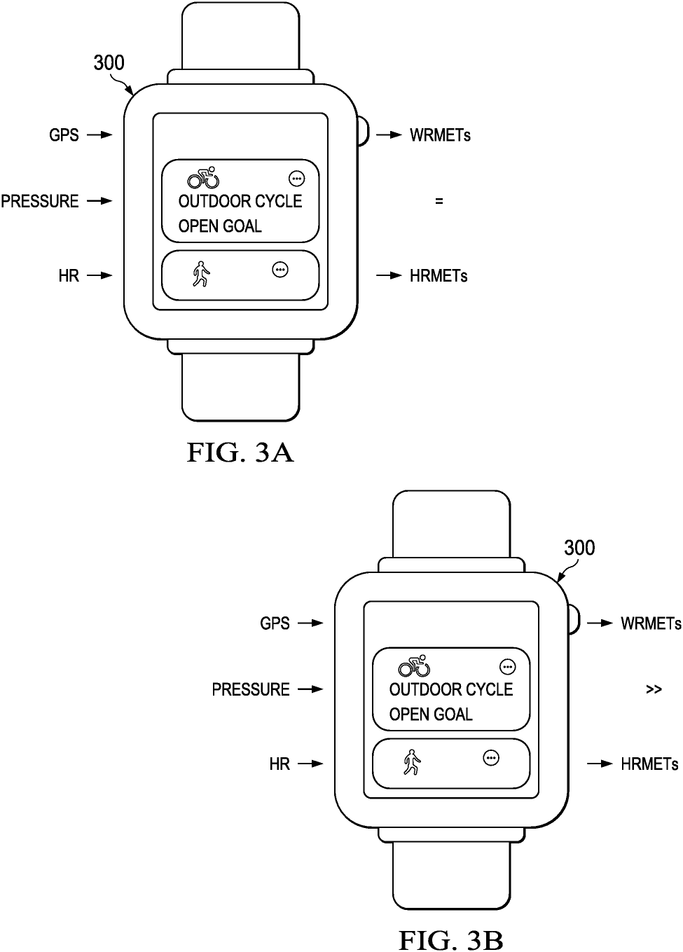

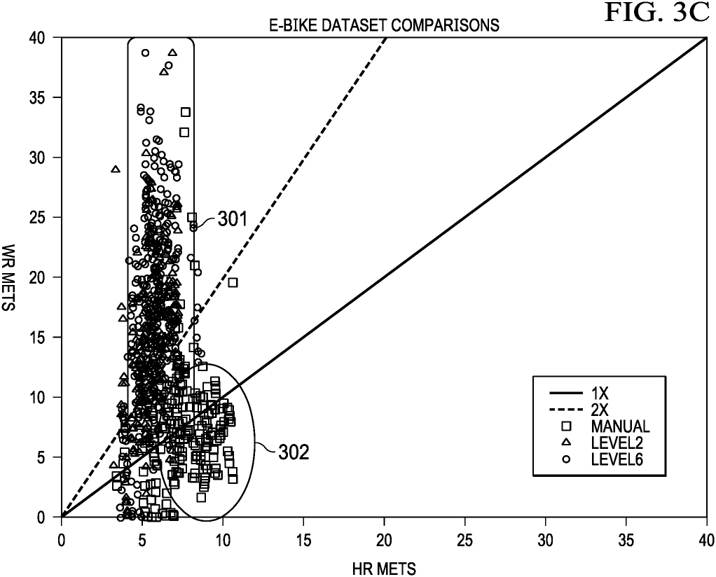

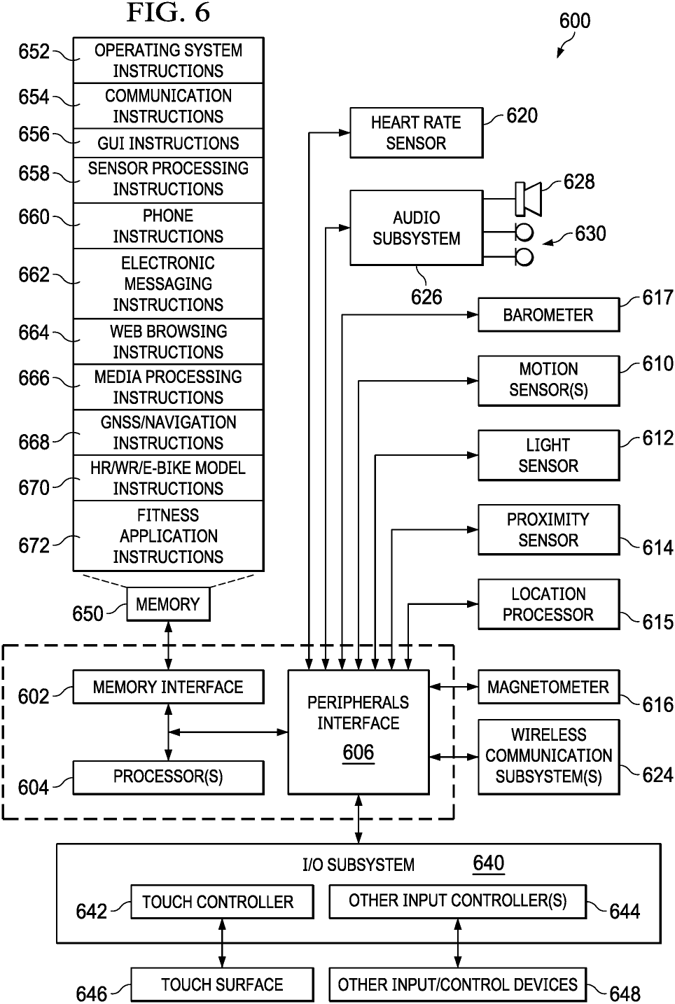

FIGS. 3A-3C illustrate the overestimation of WRMETs when a user is riding an e-bike, according to an embodiment. In the example shown, wearable device 300 is a smartwatch worn on the wrist of the user. In other embodiments, other devices, such as a fitness band, are applicable to disclosed embodiments and can be worn on the user’s wrist, waist, chest or any other body location or part based on the application and the type of wearable device 300. An example wearable device architecture is described with reference to FIG. 6.

Referring to FIGS. 3A and 3B, in an embodiment wearable device 300 includes a global navigation satellite system (GNSS) receiver, such as a GPS receiver for estimating position and speed of a user wearing wearable device 300. Wearable device 300 also includes a pressure sensor for measuring atmospheric pressure and a HR sensor for measuring the user’s HR (e.g., beats per minute). One or more processors on in wearable device 300 compute WR energy expenditure in METs (WRMETs) and HR energy expenditure in METs (HRMETs). Wearable device 300 can include a display as shown for displaying various fitness data, such as energy expenditure and user fitness goals.

Referring to FIGS. 3A and 3B, in an embodiment WRMETs can be estimated by a processor in wearable device 300 using the bicycle models shown in FIGS. 2A and 2B, and a calorimetry model as shown in Equation [1]:

$$W R M E T s = η ⋅ {P}_{t o t a l} ,$$

where Pₜₒₜₐₗ = rolling resistance + wind resistance + gravity resistance and ƞ is the user’s biomechanical efficiency. The rolling resistance, wind resistance and gravity resistance can be computed as described in U.S. Pat. Publication No. 20160058333A 1, for “Method To Estimate Physical Activity Rating From Pedometer,” filed Sep. 30, 2014, which is incorporated by reference herein in its entirety.

In an embodiment, HRMETs can be estimated by a processor in wearable device 300 using a calorimetry model with a parameterized function of fractional heart rate (FHR) according to Equation [2]:

$$H R M E T s = V {0}_{2} m a x ⋅ f \left(F H R\right) ,$$

where VO₂max is the user’s highest rate of oxygen exchange, which can initially be a default value that is calibrated by WRMETs computed by the wearable device, as described in U.S Pat. No. 10,987,006, for “Wearable Computer With Fitness Machine Connectivity For Improved Activity Monitoring Using Caloric Expenditure Models,” issued Apr. 27, 2021, which is incorporated by reference herein in its entirety.

The FHR can be computed according to Equation [3]:

$$F H R = \left(H {R}_{m a x} - H R\right) / \left(H {R}_{m a x} - R H R\right) \text{,}$$

where HR is the user’s current or measured HR, HRₘₐₓ is the user’s maximum HR and RHR is the user’s resting heart rate. The function f(FHR) may be an approximately sigmoidal nonlinearity, such that when FHR=0, f(0) may equal 1 and f(1) may equal 0, or a fractional margin above 0 (e.g., approximately 0.1, 0.2 or 0.3). Further details on computing HRMETs are disclosed in U.S. Pat. No. 10,244,948, for “Statistical Heart Rate Monitoring For Estimating Calorie Expenditure,” issued Apr. 2, 2019, which patent application is incorporated by reference herein in its entirety.

Referring to FIG. 3C, a plot of WRMETs versus HRMETs is shown for normal bike session 301 and e-bike session 302 for different power settings. The data plotted is in units of METs, which are defined as 3.5 mL of oxygen (O2) consumed per kilogram of bodyweight per minute. In FIG. 3C, the plot shown compares HRMETs based on the HR sensor data versus WRMETs based on GPS and pressure sensor data. As can be observed from the plot, WRMETs are overestimated, resulting in much larger WRMETs than HRMETs which usually track closely.

Based on the observation in FIG. 3C, an e-bike detection model was developed according to Equation [4]:

$$ln \frac{p}{1 - p} = {β}_{0} + {β}_{1} {x}_{1} + {β}_{2} {x}_{2} + {β}_{3} {x}_{3} + {β}_{4} {x}_{2} {x}_{3} ,$$

where p is the probability of being an e-bike, x₁ is a ratio between WRMETs and HRMETs and x₂ is a boolean value of “1” for being an incline or “0” for being flat and x₃ is GPS speed. In an embodiment, the coefficients β₀ to β₄ are computed using regression analysis on WRMETs and HRMETs, such as shown in FIG. 3C. In an embodiment, the coefficients are β₀=-2.83, β₁ = 78.26, β₂=-5.32, β₃=-12.76 and β₄=5.57.

In an embodiment, in addition to the probability function in Equation [4], the ratio between WRMETs and VO₂ MAX is computed and compared to a threshold to detect an e-bike. A large ratio (e.g., > 1.2) indicates a higher chance that the user is on an e-bike. A person who is riding an e-bike cannot exceed their VO₂ MAX for very long, because VO₂ MAX is the maximum energy one can produce over an extended amount of time.

The model in Equation [4] can be computed for each epoch that HRMETs and WRMETs are determined. If the probability p exceeds a threshold probability (e.g., 0.5), then the model outputs data indicating that an e-bike was detected, such as a Boolean value of “1” or sets a flag. In an embodiment, if an e-bike is detected (p > threshold) then the efficiency factor ƞ in Equation [1] is adjusted (e.g., reduced) to compensate for over estimation of WRMETs by the WRMET model.

Note that changes in the user’s effort will not have an immediate impact on HRMETs due to the user’s biology, while any speed changes (e.g., GPS speed changes) will be immediately observable. In an embodiment, the threshold probability can also be a function of at least one of contextual feedback from GPS, pressure, and inertial motion sensor sources (e.g., accelerometers).

In an embodiment, the probability p is computed over one or more sliding windows of HRMET and WRMET values (e.g., 1 minute windows), and if a percentage of the epochs in the window have p exceeding the threshold (e.g., 60%), the window is marked as having an e-bike detection. If N consecutive windows (e.g., 3 windows) are marked as having an e-bike detection, then an e-bike is detected and the efficiency factor ƞ in Equation [1] is adjusted to compensate for the over estimation of WRMETs.

### Example System

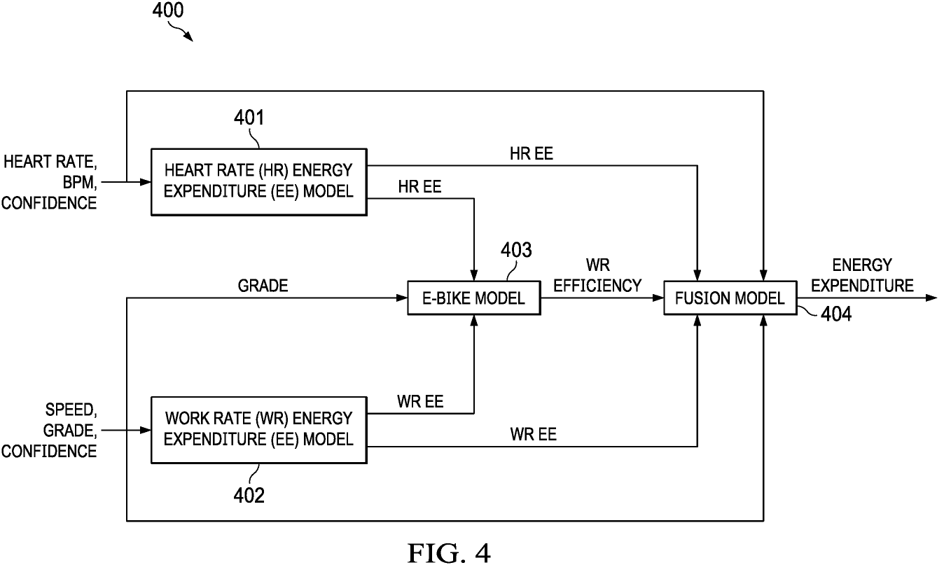

FIG. 4 is a block diagram of system 400 for determining e-bike energy expenditure, according to an embodiment. system 400 includes HR energy expenditure (EE) model 401, WR EE model 402, e-bike model 403 and fusion model 404. HR EE model 401 takes HR and other data as input and outputs HRMETs, as described above. WR EE model 402 takes speed, grade and other data as input and outputs WRMETs. The HRMET and WRMET values are input into e-bike model 403, where they are used to compute x₁ in Equation [4], i.e., the ratio of WRMET to HRMET. If an e-bike is detected as described above, then an adjusted efficiency factor ƞ is sent to fusion model 404. In other embodiments, a detection signal is sent to fusion model 404, and fusion model 404 computes and applies the adjusted efficiency factor ƞ to the WRMET to compensate for its over estimation by WR EE model 402.

### Example Process

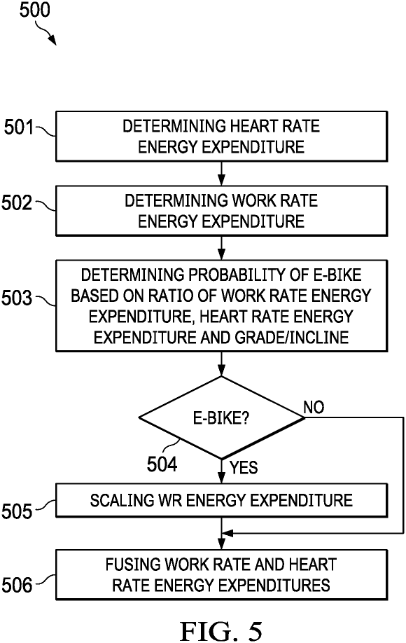

FIG. 5 is a flow diagram of a process 500 of e-bike personal energy expenditure, according to an embodiment. Process 500 can be implemented using the wearable device architecture 600 disclosed in reference to FIG. 6.

Process 500 begins by determining heart rate energy expenditure (501). For example, heart rate energy expenditure can be determined as described with reference to FIG. 3C using a heart rate sensor.

Process 500 continues by determining work rate energy expenditure (502). For example, heart rate energy expenditure can be determined as described with reference to FIG. 3C using a heart rate sensor.

Process 500 continues by determining a probability that the user is riding an e-bike based on a ratio of work rate energy expenditure, heart rate energy expenditure and incline data (503). Incline data can be determined by motion sensors and/or the pressure sensor.

Process 500 continues by determining, based on the probability, if the user is riding an e-bike (504). For example, the probability can be computed according to Equation [4] and compared to a threshold probability (e.g., 0.5) and if the probability meets a condition corresponding to the threshold probability (e.g., exceeds that threshold probability) an e-bike is detected. In an embodiment, the probability p is computed over one or more sliding windows of HR/WR MET values (e.g., 1 minute windows), and if a percentage of the epochs in the window have p exceeding the threshold probability (e.g., 60%), the window is marked as having an e-bike detection. If N consecutive windows (e.g., 3 windows) are marked as having an e-bike detection, then an e-bike is detected and the efficiency factor ƞ is adjusted to compensate for the over estimation of WR EE by the WR EE model.

Note that other embodiments may use different heart rate and work rate energy expenditure models (e.g., based on VO₂ max), and adjusting the work rate energy expenditure can be done in any suitable manner based on the particular work rate and/or heart rate energy expenditure model used.

In accordance with an e-bike being detected (504), the work rate energy expenditure is scaled (505) to compensate for over estimation, and the scaled work rate energy expenditure and heart rate energy expenditure are fused (e.g., averaged) to determine a final energy expenditure for the user (506). For example, an efficiency factor can be used to reduce the work rate energy expenditure as shown in Equation [1].

In accordance with an e-bike not being detected (504), the work rate energy expenditure and heart rate energy expenditure are fused without adjusting the efficiency factor based on e-bike detection (506).

Fitness data or any other data can be generated based at least on the adjusted work rate energy expenditure or the fused work rate energy expenditure and heart rate energy expenditure. The data can be, for example, displayed or stored on the wearable device, or transmitted to another device or network for further processing or storage. The data can be used as is by a fitness application or other application running on the wearable device and/or another device and or can be used to compute a variety of fitness data and charts, such as calories burned by the user over various time periods, personal comparative performance metrics and the like.

### Exemplary Wearable Device Architecture

FIG. 6 illustrates example wearable device architecture 600 implementing the features and operations described in reference to FIGS. 1-5. Architecture 600 can include memory interface 602, one or more hardware data processors, image processors and/or processors 604 and peripherals interface 606. Memory interface 602, one or more processors 604 and/or peripherals interface 606 can be separate components or can be integrated in one or more integrated circuits.

Sensors, devices and subsystems can be coupled to peripherals interface 606 to provide multiple functionalities. For example, one or more motion sensors 610, light sensor 612 and proximity sensor 614 can be coupled to peripherals interface 606 to facilitate motion sensing (e.g., acceleration, rotation rates), lighting and proximity functions of the wearable device. Location processor 615 can be connected to peripherals interface 606 to provide geo-positioning. In some implementations, location processor 615 can be a GNSS receiver, such as the Global Positioning System (GPS) receiver. Electronic magnetometer 616 (e.g., an integrated circuit chip) can also be connected to peripherals interface 606 to provide data that can be used to determine the direction of magnetic North. Electronic magnetometer 616 can provide data to an electronic compass application. Motion sensor(s) 610 can include one or more accelerometers and/or gyros configured to determine change of speed and direction of movement of the wearable device. Pressure sensor 617 (e.g., a barometer) can be configured to measure atmospheric pressure around the mobile device.

Heart rate monitoring subsystem 620 for measuring the heartbeat of a user who is wearing the device on their wrist. In an embodiment, subsystem 620 includes a PPG to detect a heartbeat.

Communication functions can be facilitated through wireless communication subsystems 624, which can include radio frequency (RF) receivers and transmitters (or transceivers) and/or optical (e.g., infrared) receivers and transmitters. The specific design and implementation of the communication subsystem 624 can depend on the communication network(s) over which a mobile device is intended to operate. For example, architecture 600 can include communication subsystems 624 designed to operate over a GSM network, a GPRS network, an EDGE network, a Wi-Fi™ network and a Bluetooth™ network. In particular, the wireless communication subsystems 624 can include hosting protocols, such that the mobile device can be configured as a base station for other wireless devices.

Audio subsystem 626 can be coupled to a speaker 628 and a microphone 630 to facilitate voice-enabled functions, such as voice recognition, voice replication, digital recording and telephony functions. Audio subsystem 626 can be configured to receive voice commands from the user.

I/O subsystem 640 can include touch surface controller 642 and/or other input controller(s) 644. Touch surface controller 642 can be coupled to a touch surface 646. Touch surface 646 and touch surface controller 642 can, for example, detect contact and movement or break thereof using any of a plurality of touch sensitivity technologies, including but not limited to capacitive, resistive, infrared and surface acoustic wave technologies, as well as other proximity sensor arrays or other elements for determining one or more points of contact with touch surface 646. Touch surface 646 can include, for example, a touch screen or the digital crown of a smart watch. I/O subsystem 640 can include a haptic engine or device for providing haptic feedback (e.g., vibration) in response to commands from processor 604. In an embodiment, touch surface 646 can be a pressure-sensitive surface.

Other input controller(s) 644 can be coupled to other input/control devices 648, such as one or more buttons, rocker switches, thumb-wheel, infrared port and USB port. The one or more buttons (not shown) can include an up/down button for volume control of speaker 628 and/or microphone 630. Touch surface 646 or other controllers 644 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 646; and a pressing of the button for a second duration that is longer than the first duration may turn power to the mobile device on or off. The user may be able to customize a functionality of one or more of the buttons. The touch surface 646 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 602 can be coupled to memory 650. Memory 650 can include high-speed random access memory and/or non-volatile memory, such as one or more magnetic disk storage devices, one or more optical storage devices and/or flash memory (e.g., NAND, NOR). Memory 650 can store operating system 652, such as the iOS operating system developed by Apple Inc. of Cupertino, California. Operating system 652 may include instructions for handling basic system services and for performing hardware dependent tasks. In some implementations, operating system 652 can include a kernel (e.g., UNIX kernel).

Memory 650 may also store communication instructions 654 to facilitate communicating with one or more additional devices, one or more computers and/or one or more servers, such as, for example, instructions for implementing a software stack for wired or wireless communications with other devices. Memory 650 may include graphical user interface instructions 656 to facilitate graphic user interface processing; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GNSS/Location instructions 668 to facilitate generic GNSS and location-related processes and instructions; and HR/WR models and e-bike detection instructions 670 to facilitate e-bike detection and compensating WR EE for e-bikes, as described in reference to FIGS. 1-5. Memory 650 further includes fitness application instructions for using the compensated WR EE to calculate calories burned and/or any other energy expenditure or fitness metric.

Each of the above identified instructions and applications can correspond to a set of instructions for performing one or more functions described above. These instructions need not be implemented as separate software programs, procedures, or modules. Memory 650 can include additional instructions or fewer instructions. Furthermore, various functions of the mobile device may be implemented in hardware and/or in software, including in one or more signal processing and/or application specific integrated circuits.

The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language (e.g., SWIFT, Objective-C, C#, Java), including compiled or interpreted languages, and it can be deployed in any form, including as a stand-alone program or as a module, component, subroutine, a browser-based web application, or other unit suitable for use in a computing environment.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

1. A method comprising:
determining, with the least one processor of a wearable device, heart rate energy expenditure of a user wearing or holding the device;
determining, with at least one processor of a wearable device, work rate energy expenditure of a user wearing or holding the device;
determining, with the at least one processor, a probability that the user is riding an electrical bike based on the heart rate energy expenditure and the work rate energy expenditure;
determining, with the at least one processor, whether or not the probability meets a condition corresponding to a threshold probability; and
in accordance with the probability meeting the condition corresponding to the threshold probability:
adjusting, with the at least one processor, the work rate energy expenditure; and
generating, with the at least one processor, fitness data based at least on the adjusted work rate energy expenditure.

2. The method of claim 1, wherein the device is a smartwatch of fitness band worn on the wrist of the user.

3. The method of claim 1, wherein determining a probability that the user is riding an electrical bicycle based on the heart rate energy expenditure and the work rate energy expenditure includes evaluating a probability function that includes a ratio of work rate energy expenditure and heart rate energy expenditure.

4. The method of claim 3, wherein the probability function is also a function of incline data indicating that the user is riding a bike or electrical bike on an incline.

5. The method of claim 4, wherein the probability function is given by:
$$\text{ln} \frac{p}{1 - p} \text{=} {β}_{0} + {β}_{1} {x}_{1} + {β}_{2} {x}_{2} + {β}_{3} {x}_{1} {x}_{2} ,$$
where p is the probability of being an electrical bicycle, x₁ is the ratio between work rate energy expenditure and heart rate energy expenditure and x₂ is a boolean value of “1” for being an incline or “0” for being flat.

6. The method of claim 3, wherein determining whether or not the probability meets a condition corresponding to a threshold probability, further comprises:
computing the probability over one or more sliding windows of work rate energy expenditure and heart rate energy expenditure values; and
determining a percentage of measurement epochs in the one or more windows having a probability that meets the condition corresponding to the threshold probability.

7. The method of claim 6, further comprising:
determining, with the at least one processor, that an electrical bicycle is detected based on two or more sliding windows having a percentage of measurement epochs having a probability that meets the condition corresponding to the threshold probability.

8. An apparatus comprising:
a global navigation satellite system (GNSS) configured to estimate speed of a user wearing or holding the apparatus;
at least one heart rate sensor;
one or more processors;
memory storing instructions that when executed by the one or more processors, cause the one or more processors to perform operations comprising:
determining, using the at least one heart rate sensor, heart rate energy expenditure of the user;
determining work rate energy expenditure of the user based on the speed of the user;
determining a probability that the user is riding an electrical bike based on the heart rate energy expenditure and the work rate energy expenditure of the user;
determining whether or not the probability meets a condition corresponding to a threshold probability; and
in accordance with the probability meeting the condition corresponding to the threshold probability:
adjusting the work rate energy expenditure; and
generating fitness data for the user based at least on the adjusted work rate energy expenditure.

9. The apparatus of claim 8, wherein the device is a smartwatch or fitness band worn on the wrist of the user or other body part.

10. The apparatus of claim 8, wherein determining a probability that the user is riding an electrical bicycle based on the heart rate energy expenditure and the work rate energy expenditure includes evaluating a probability function that includes a ratio of work rate energy expenditure and heart rate energy expenditure.

11. The apparatus of claim 10, wherein the probability function is also a function of incline data indicating that the user is riding a bike or electrical bike on an incline.

12. The apparatus of claim 11, wherein the probability function is given by:
$$ln \frac{p}{1 - p} = {β}_{0} + {β}_{1} {x}_{1} + {β}_{2} {x}_{2} + {β}_{3} {x}_{1} {x}_{2} ,$$
where p is the probability of being an electrical bicycle, x₁ is the ratio between work rate energy expenditure and heart rate energy expenditure and x₂ is a boolean value of “1” for being an incline or “0” for being flat.

13. The apparatus of claim 10, wherein determining whether or not the probability meets a condition corresponding to a threshold probability, further comprises:
computing the probability over one or more sliding windows of work rate energy expenditure and heart rate energy expenditure values; and
determining a percentage of measurement epochs in the one or more windows having a probability that meets the condition corresponding to the threshold probability.

14. The apparatus of claim 13, the operations further comprising:
determining, with the at least one processor, that an electrical bicycle is detected based on two or more sliding windows having a percentage of measurement epochs having a probability that meets the condition corresponding to the threshold probability.

15. A non-transitory, computer-readable storage medium having stored thereon instructions that when executed by at least one processor of a wearable device, cause the at least one processor to perform operations comprising:
determining heart rate energy expenditure of a user wearing or holding the device;
determining work rate energy expenditure of a user wearing or holding the device;
determining a probability that the user is riding an electrical bike based on the heart rate energy expenditure and the work rate energy expenditure;
determining whether or not the probability meets a condition corresponding to a threshold probability; and
in accordance with the probability meeting the condition corresponding to the threshold probability:
adjusting work rate energy expenditure; and
generating fitness data based at least on the adjusted work rate energy expenditure.

16. The non-transitory, computer-readable storage medium of claim 15, wherein determining a probability that the user is riding an electrical bicycle based on the heart rate energy expenditure and the work rate energy expenditure includes evaluating a probability function that includes a ratio of work rate energy expenditure and heart rate energy expenditure.

17. The non-transitory, computer-readable storage medium of claim 16, wherein the probability function is also a function of incline data indicating that the user is riding a bike or electrical bike on an incline.

18. The non-transitory, computer-readable storage medium of claim 17, wherein the probability function is given by:
$$ln \frac{p}{1 - p} = {β}_{0} + {β}_{1} {x}_{1} + {β}_{2} {x}_{2} + {β}_{3} {x}_{1} {x}_{2} ,$$
where p is the probability of being an electrical bicycle, x₁ is the ratio between work rate energy expenditure and heart rate energy expenditure and x₂ is a boolean value of “1” for being an incline or “0” for being flat.

19. The non-transitory, computer-readable storage medium of claim 16, wherein determining whether or not the probability meets a condition corresponding to a threshold probability, further comprises:
computing the probability over one or more sliding windows of work rate energy expenditure and heart rate energy expenditure values; and
determining a percentage of measurement epochs in the one or more windows having a probability that meets the condition corresponding to the threshold probability.

20. The non-transitory, computer-readable storage medium of claim 19, further comprising:
determining, with the at least one processor, that an electrical bicycle is detected based on two or more sliding windows having a percentage of measurement epochs having a probability that meets the condition corresponding to the threshold probability.
